(19) United States
(12) Patent Application Publication
(10) Pub. No.: US 2005/0004104 A1
(43) Pub. Date: Jan. 6, 2005

(54) Methods for the protection of memory and cognition

(76) Inventors: Brian M. Cali, Arlington, MA (US); Yueh-Tyng Chien, Newton, MA (US); Mark G. Currie, Sterling, MA (US); John Jeffrey Talley, Somerville, MA (US); Craig Zimmerman, Topsfield, MA (US)

Correspondence Address: FISH & RICHARDSON PC, 225 FRANKLIN ST, BOSTON, MA, 02110, US

(21) Appl. No.: 10/859,335

(22) Filed: Jun. 1, 2004

Related U.S. Application Data
(60) Provisional application No. 60/475,204, filed on May 30, 2003.

(51) Int. Cl. A61K 31/55; A61K 31/454; A61K 31/405

(52) U.S. Cl. 514/217.09; 514/419; 514/323; 514/414

(57) ABSTRACT

The invention features certain compounds useful in the treatment of memory disorders, i.e., they reduce or delay memory loss or they enhance memory retention. Because certain of the compounds do not substantially inhibit either COX-1 or COX-2 at therapeutically relevant doses, these compounds are far less likely to cause gastrointestinal ulceration than is indomethacin, which is known to inhibit both COX-1 and COX-2. Certain of the compounds inhibit the activity of DAO at therapeutically relevant doses. Among the memory disorders that can be treated are AD, mild cognitive impairment (MCI; a common precursor to AD), and memory loss or cognitive impairment associated with vascular dementias, amnesia, dementia, AIDS dementia, Huntington's Disease, hydrocephalus, depression, Pick's Disease, Creutzfeldt-Jakob Syndrome, electroconvulsive therapy, or Parkinson's Disease.

### CLAIM OF PRIORITY

This application claims priority under 35 USC § 119(e) to U.S. Patent Application Ser. No. 60/475,204, filed on May 30, 2003, the entire contents of which is hereby incorporated by reference.

### TECHNICAL FIELD

This invention relates to methods for the treatment and prevention of cognitive impairment, e.g., memory loss, and for the enhancement of cognitive function and memory.

### BACKGROUND

Cognitive impairment and memory loss are associated with a number of disorders and conditions, including mild cognitive impairment, amnesia, Alzheimer's Disease (AD), vascular dementias, AIDS dementia, dementia, Huntington's Disease, hydrocephalus, depression, Pick's Disease, Creutzfeldt-Jakob Syndrome, electroconvulsive therapy, and Parkinson's Disease.

There are currently several approved therapies treatment of memory loss, including memory loss associated with Alzheimer's Disease (AD). Among the approved therapies several, tacrine (Cognex®), donepezil hydrochloride (Aricept®), galantamine (Reminyl®), and rivastigmine (Exelon®), are thought to act by inhibiting cholinesterase. Other potentially useful agents include NMDA receptor antagonists (e.g., memantine), M1 muscammic receptor antagonists, vitamin E/tocopherol, statins (e.g., lovastatin, pravastatin (Pravachol®, Bristol-Meyers Squibb, Lawrenceville, N.J.)), CX516 (Ampalex®; Cortex Pharmaceuticals, Irvine, Calif.), aripipazole (Bristol-Meyers Squibb, Lawrenceville, N.J.), CPI-1189 (Centaur Pharmaceuticals, Sunnyvale, Calif.), leteprinim potassium (Neotrofin®; NeoTherapeutics, Inrine, Calif.), phenserine tartrate (Axonyx, New York, N.Y.), conjugated estrogen (Premain®; Wyeth, Philadelphia, Pa.), risperidone (Risperdal®, Johnson & Johnson Pharmaceutcals Research and Development, Raritan, N.J.), SB271046 (GlaxoSmithKline, Philadelphia, Pa.), SB737552 (GlaxoSmithKline, Philadelphia, Pa.), SR 57667 (Sanofi-Synthelabo, New York, N.Y.), and SR 57746 (Sanofi-Synthelabo, New York, N.Y.). Epidemiological studies indicate that the use of non-steroidal anti-inflammatory drugs (NSAIDs) is associated with a reduced risk of developing AD. Numerous clinical studies reported a negative association between NSAID use and the incidence of AD (McGeer et al. 1996 Neurology 47:425; Akiyama et al. 2000 Neurobiol Aging 21:283). The primary action of NSAIDs is inhibition of the cyclooxygenase enzymes, COX-1, which is constitutive, and COX-2, which is inducible. COX-1 and COX-2 are involved in the biosynthesis of prostaglandins and thromboxanes. Both COX-1 and COX-2 are thought to be involved in numerous inflammatory responses and in normal neuronal function. It has been proposed that the beneficial effects of NSAIDS relative to the development of AD are associated with the anti-inflammatory effects of NSAIDs via the inhibition of COX-1 and or COX-2 (McGeer 2000 Drugs Aging 17:1). In vitro studies have shown the non-selective COX inhibitors can preferentially decrease the levels of the highly amyloidogenic amyloid-beta (Aβ)(1-42) peptide. Studies on the effect of NSAIDs in a murine model of AD neuropathology suggest that the frequency of Aβ plaque deposits in the brains of these animals can be significantly reduced by treatment with the non-selective COX inhibitor ibuprofen. Since AD patients routinely present with inflammatory changes in the brain (Aisen and Davis 1994 Am J Psychiatr 151:1105), NSAIDs that can cross the blood-brain barrier have been suggested as potentially useful for reducing brain inflammation.

Indomethacin (I) (1-(4-chlorobenzoyl)-5-methoxy-2-methyl-1H-indole-3-acetic acid), a NSAID, has been shown to improve, in an acute treatment setting, sensorimotor coordination and short-term memory in healthy elderly volunteers (Bruce-Jones et al. 1994 Br J Clin Pharmacol 38:45). Moreover, standard anti-inflammatory doses of indomethacin have been shown to protect against further cognitive decline in AD patients with mild to moderate memory impairment (Rogers et al. 1993 Neurology 43:1609) in both an acute and chronic dosing manner. In preclinical studies, indomethacin was shown to significantly reduce recall latency in a rat model of retrograde amnesia (Rao et al. 2002 Biol Psychiatry 51:770). These results have been attributed to the actions of indomethacin on cyclooxygenase, the enzyme target for the NSAID activity of this drug (Rao et al.). It has also been reported that indomethacin, along with ibuprofen and sulindac sulphide, significantly decreases the amyloidogenic Aβ42 peptide produced from a variety of cultured cells and that this effect might not be caused by COX inhibition (Weggen et al. 2001 Nature 414: 212).

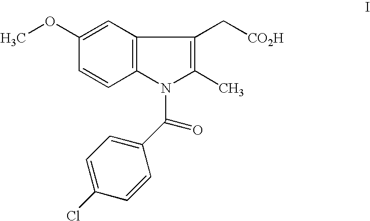

There is also some evidence that indomethacin and other NSAIDs may impair memory of cognitive ability. Based on a review of the literature, Hoppmann et al. (Arch Intern Med 151:1309, 1991) concluded that central nervous system side effects of the NSAIDs include aseptic meningitis, psychosis, and cognitive dysfunction. They concluded that psychosis, although infrequently reported with NSAIDs, should be suspected in an elderly patient started on a regimen of indomethacin who acutely develops disorientation, paranoia, or hallucinations and that there appears to be some potential for memory dysfunction and attention deficits in elderly patients treated with NSAIDs.

More recently, Teather et al. (Learning and Memory 9:41, 2002) tested the effect of indomethacin, which is a non-selective COX inhibitor, and NS-398, a COX-2 specific inhibitor, on memory using two different rat memory models. One model tested the effect of the compounds on cognitive memory and the other model tested the effect of the compounds on stimulus-response habit formation. Teather et al. found that COX-2 specific inhibitors, and indomethacin, inhibited consolidation of hippocampus-dependent cognitive memory.

It has been suggested that certain inhibitors of D-amino oxidase (DAO), including certain heterocylc-2-carboxylic acids, might be useful for improving memory, learning and cognition in patients suffering from neurodegenerative disorders (U.S. Patent Application Publication U.S. 2003/0162825 A1). Indomethacin has also been shown to be an inhibitor of DAO (Chen et. al 1994 Drug Metabol Drug Interact. 11:153-60). DAO degrades D-serine and other D-amino acids. D-glutamate and D-serine are thought to be agonists of N-methyl-D-aspartate (NMDA)-glutamate receptors that mediate a wide variety of brain activities, including the synaptic plasticity that is associated with certain types of memory and learning (U.S. Pat. No. 20030162825 A1). Thus, it is thought that inhibition of DAO will lead to increased D-serine levels and improved cognitive function.

### SUMMARY

The invention features certain compounds useful in the treatment of memory disorders, i.e., they reduce or delay memory loss or they enhance memory retention. Because certain of the compounds do not substantially inhibit either COX-1 or COX-2 at therapeutically relevant doses, these compounds are far less likely to cause gastrointestinal ulceration than is indomethacin, which is known to inhibit both COX-1 and COX-2. Certain of the compounds inhibit the activity of DAO at therapeutically relevant doses. Among the memory disorders that can be treated are AD, mild cognitive impairment (MCI; a common precursor to AD), and memory loss or cognitive impairment associated with vascular dementias, amnesia, dementia, AIDS dementia, Huntington's Disease, hydrocephalus, depression, Pick's Disease, Creutzfeldt-Jakob Syndrome, electroconvulsive therapy, or Parkinson's Disease.

The compounds can also be used to enhance memory or cognitive function, e.g., in patients that are not suffering from a disorder associated with memory loss or impairment of cognitive function.

In one aspect the invention features a method for treating a patient comprising administering a compound having the formula (II): 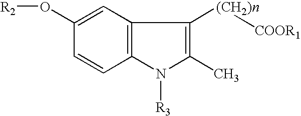
wherein:
R1 is H or an independently substituted or unsubstituted C1-C10 alkyl, C3-C8 cycloalkyl, arylalkyl or heteroarylalkyl wherein the substituents are selected from the group consisting of amino, halogen and hydroxy;
n=1, 2, or 3; wherein each CH₂ within (CH₂)ₙ can be optionally independently substituted with one or more substituents selected from methyl, halogen and hydroxy;
R₂ is H or an independently substituted or unsubstituted C1-C4 alkyl or C3-C5 cycloalkyl wherein the substituents are selected from the group consisting of: methyl, halogen and hydroxy;
R₃ is H, —CH₂R₄, —C(O)R₄, —SO₂R₄, —CONR₅R₆ wherein:
R₄ is an independently substituted unsubstituted aryl or heteroaryl wherein the substituents are selected from halogen, OCH₃, OCF₂H, OCF₃, CH₃, CN, CF₂H, CF₃, SCH₃, SCF₃,
R₅ and R₆ are independently H, C1-C6 independently substituted or unsubstituted alkyl or R₅ together with R₆ form a 3 to 7-membered carbocyclic or heterocyclic ring wherein the heteroatoms are selected from O, S, SO, SO₂ and NR₇ wherein R₇ is H or an independently substituted or unsubstituted C₁-C3 alkyl, wherein the substituents are selected from methyl, amino, halogen and hydroxy; and
(b) a pharmaceutically acceptable carrier.

In various embodiments: n is 1; n is 2; R₁ is H; R1 is C1-C3 alkyl; R₁ is methyl; R₃ is H; R₄ is a phenyl group; R₄ is unsubstituted; R₄ is substituted; R₃ is —CH₂R₄, or —C(O)R₄; R₄ is a phenyl group; the phenyl group is a substituted phenyl group; the phenyl group is independently substituted at the 3 and 4 positions; phenyl group is independently substituted at the 2 and 4 positions; the phenyl group is independently substituted at the 2 and 3 positions; phenyl group is substituted at the 4 position; phenyl group is substituted at the 3 position; the phenyl group is independently substituted with one or more halogens; phenyl group is substituted at the 4 position with CF₃; the patient is suffering from one or more disorders chosen from short term memory, loss of long term memory, Alzheimer's Disease, and mild cognitive impairment; the patient is suffering from or at risk of developing impairment of cognitive function associated with treatment with a therapeutic agent; the patient is suffering from one or more disorders chosen from: vascular dementia, Huntington's Disease, hydrocephalus, depression, amnesia, AIDS-related dementia, Pick's Disease, Creutzfeldt-Jakob Syndrome, and Parkinson's Disease; the patient has undergone electroconvulsive therapy; the compound is not: 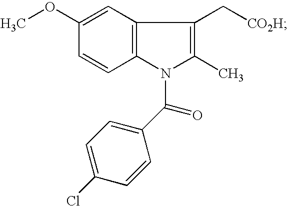
method further includes administering an agent chosen from: tacrine, donepezil hydrochloride, galantamine, rivastigmine, a cholinesterase inhibitor, an NMDA receptor antagonist, a M1 muscarinic receptor antagonist, vitamin E/tocopherol, a statin, CX516, aripipazole, CPI-1189, leteprinim potassium, phenserine tartrate, pravastatin, conjugated estrogen, risperidone, SB737552, SR 57667, and SR 57746; the compound does not substantially inhibit COX-1 activity; the compound does not substantially inhibit COX-2 activity; the compound does not substantially inhibit COX-1 activity or COX-2 activity.

In certain embodiments the compound is selected from:
(5-methoxy-2-methyl-1H-indol-3-yl)acetic acid;
(5-hydroxy-2-methyl-1H-indol-3-yl)acetic acid;
(1-benzyl-5-hydroxy-2-methyl-1H-indol-3-yl)acetic acid;
[1-(3,4-dichlorobenzoyl)-5-hydroxy-2-methyl-1H-indol-3-yl]acetic acid;
[1-(3-chlorobenzoyl)-5-methoxy-2-methyl-1H-indol-3-yl]acetic acid;
[1-(3-chlorobenzyl)-5-methoxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
(1-benzyl-5-hydroxy-2-methyl-1H-indol-3-yl)acetic acid;
[1-(4-chlorobenzyl)-5-hydroxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
{5-hydroxy-2-methyl-1-[4-(trifluoromethyl)benzoyl]-1H-indol-3-yl}acetic acid;
[1-(2,4-dichlorobenzoyl)-5-hydroxy-2-methyl-1H-indol-3-yl]acetic acid;
[1-(2,3-dichlorobenzoyl)-5-methoxy-2-methyl-1H-indol-3-yl]acetic acid;
[1-(2,3-dichlorobenzoyl)-5-hydroxy-2-methyl-1H-indol-3-yl]acetic acid;
{1-[(6-chloropyridin-3-yl)carbonyl]-5-hydroxy-2-methyl-1H-indol-3-yl}acetic acid;
[1-(3-chlorobenzoyl)-5-methoxy-2-methyl-1H-indol-3-yl]acetic acid;
[1-(3-chlorobenzoyl)-5-hydroxy-2-methyl-1H-indol-3-yl]acetic acid;
[1-(3,4-difluorobenzoyl)-5-methoxy-2-methyl-1H-indol-3-yl]acetic acid;
[1-(3,4-difluorobenzoyl)-5-hydroxy-2-methyl-1H-indol-3-yl]acetic acid;
[1-(4-bromobenzyl)-5-hydroxy-2-methyl-1H-indol-3-yl]acetic acid;
[1-(4-chlorobenzyl)-5-methoxy-2-methyl-1H-indol-3-yl]acetic acid;
[1-(4-fluorobenzyl)-5-methoxy-2-methyl-1H-indol-3-yl]acetic acid;
[5-hydroxy-2-methyl-1-(3-methylbenzoyl)-1H-indol-3-yl]acetic acid;
{1-[(4-chlorophenyl)sulfonyl]-5-methoxy-2-methyl-1H-indol-3-yl}acetic acid;
{1-[(4-chlorophenyl)sulfonyl]-5-hydroxy-2-methyl-1H-indol-3-yl}acetic acid;
[5-methoxy-2-methyl-1-(piperidin-1-ylcarbonyl)-1H-indol-3-yl]acetic acid;
[5-hydroxy-2-methyl-1-(3-phenylprop-2-ynoyl)-1H-indol-3-yl]acetic acid;
propyl (5-hydroxy-2-methyl-1H-indol-3-yl)acetate; and
ethyl[1-(4-chlorobenzoyl)-5-methoxy-2-methyl-1H-indol-3-yl] acetate.

The invention also includes a pharmaceutical composition comprising:
(a) a compound having the formula: 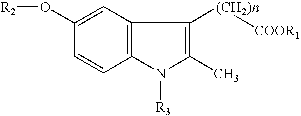
   wherein:
R₁ is H or an independently substituted or unsubstituted C1-C10 alkyl, C3-C8 cycloalkyl, arylalkyl or heteroarylalkyl wherein the substituents are selected from the group consisting of amino, halogen and hydroxy;
n=1, 2, or 3; wherein each CH₂ within (CH₂)ₙ can be optionally independently substituted with one or more substituents selected from methyl, halogen and hydroxy;
R₂ is H or an independently substituted or unsubstituted C1-C4 alkyl or C3-C5 cycloalkyl wherein the substituents are selected from the group consisting of: methyl, halogen and hydroxy;
R₃ is H, —CH₂R₄, —C(O)R₄, —SO₂R₄, —CONR₅R₆ wherein:
R₄ is an independently substituted unsubstituted aryl or heteroaryl wherein the substituents are selected from halogen, OCH₃, OCF₂H, OCF₃, CH₃, CN, CF₂H, CF₃, SCH₃, SCF₃,
R₅ and R₆ are independently H, C1-C6 independently substituted or unsubstituted alkyl or R₅ together with R₆ form a 3 to 7-membered carbocyclic or heterocyclic ring wherein the heteroatoms are selected from O, S, SO, SO₂ and NR₇ wherein R₇ is H or an independently substituted or unsubstituted C1-C3 alkyl, wherein the substituents are selected from methyl, amino, halogen and hydroxy; and
(b) a pharmaceutically acceptable carrier. 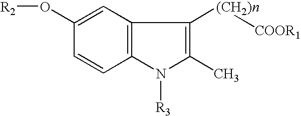
   wherein:
R₁ is H or an independently substituted or unsubstituted C1-C10 alkyl, C3-C8 cycloalkyl, arylalkyl or heteroarylalkyl wherein the substituents are selected from the group consisting of amino, halogen and hydroxy;
n=1, 2, or 3; wherein each CH₂ within (CH₂)ₙ can be optionally independently substituted with one or more substituents selected from methyl, halogen and hydroxy;
R₂ is H or an independently substituted or unsubstituted C1-C4 alkyl or C3-C5 cycloalkyl wherein the substituents are selected from the group consisting of: methyl, halogen and hydroxy;
R₃ is H, —CH₂R₄, —C(O)R₄, —SO₂R₄, —CONR₅R₆ wherein:
R₄ is an independently substituted unsubstituted aryl or heteroaryl wherein the substituents are selected from halogen, OCH₃, OCF₂H, OCF₃, CH₃, CN, CF₂H, CF₃, SCH₃, SCF₃,
R₅ and R₆ are independently H, C1-C6 independently substituted or unsubstituted alkyl or R₅ together with R6 form a 3 to 7-membered carbocyclic or heterocyclic ring wherein the heteroatoms are selected from O, S, SO, SO₂ and NR₇ wherein R₇ is H or an independently substituted or unsubstituted C1-C3 alkyl, wherein the substituents are selected from methyl, amino, halogen and hydroxy; and
(b) a pharmaceutically acceptable carrier.

In various embodiments: n is 1; n is 2; R₁ is H; R₁ is C1-C3 alkyl; R₁ is methyl; R₃ is H; R₄ is a phenyl group; R₄ is unsubstituted; R₄ is substituted; R₃ is —CH₂R₄, or —C(O)R₄; R₄ is a phenyl group; the phenyl group is a substituted phenyl group; the phenyl group is independently substituted at the 3 and 4 positions; phenyl group is independently substituted at the 2 and 4 positions; the phenyl group is independently substituted at the 2 and 3 positions; phenyl group is substituted at the 4 position; phenyl group is substituted at the 3 position; the phenyl group is independently substituted with one or more halogens; phenyl group is substituted at the 4 position with CF₃; the compound is not: 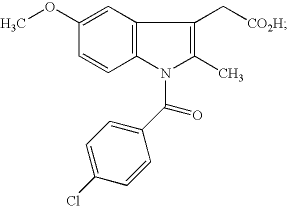
composition further includes administering an agent chosen from: tacrine, donepezil hydrochloride, galantamine, rivastigmine, a cholinesterase inhibitor, an NMDA receptor antagonist, a M1 muscarinic receptor antagonist, vitamin E/tocopherol, a statin, CX516, aripipazole, CPI-1189, leteprinim potassium, phenserine tartrate, pravastatin, conjugated estrogen, risperidone, SB737552, SR 57667, and SR 57746; the compound does not substantially inhibit COX-1 activity; the compound does not substantially inhibit COX-2 activity; the compound does not substantially inhibit COX-1 activity or COX-2 activity.

In certain embodiments the compound is selected from:
(5-methoxy-2-methyl-1H-indol-3-yl)acetic acid;
(5-hydroxy-2-methyl-1H-indol-3-yl)acetic acid;
(1-benzyl-5-hydroxy-2-methyl-1H-indol-3-yl)acetic acid;
[1-(3,4-dichlorobenzoyl)-5-hydroxy-2-methyl-1H-indol-3-yl]acetic acid;
[1-(3-chlorobenzoyl)-5-methoxy-2-methyl-1H-indol-3-yl]acetic acid;
[1-(3-chlorobenzyl)-5-methoxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
(1-benzyl-5-hydroxy-2-methyl-1H-indol-3-yl)acetic acid;
[1-(4-chlorobenzyl)-5-hydroxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
{5-hydroxy-2-methyl-1-[4-(trifluoromethyl)benzoyl]-1H-indol-3-yl}acetic acid;
[1-(2,4-dichlorobenzoyl)-5-hydroxy-2-methyl-1H-indol-3-yl]acetic acid;
[1-(2,3-dichlorobenzoyl)-5-methoxy-2-methyl-1H-indol-3-yl]acetic acid;
[1-(2,3-dichlorobenzoyl)-5-hydroxy-2-methyl-1H-indol-3-yl]acetic acid;
{1-[(6-chloropyridin-3-yl)carbonyl]-5-hydroxy-2-methyl-1H-indol-3-yl}acetic acid;
[1-(3-chlorobenzoyl)-5-methoxy-2-methyl-1H-indol-3-yl]acetic acid;
[1-(3-chlorobenzoyl)-5-hydroxy-2-methyl-1H-indol-3-yl]acetic acid;
[1-(3,4-difluorobenzoyl)-5-methoxy-2-methyl-1H-indol-3-yl]acetic acid;
[1-(3,4-difluorobenzoyl)-5-hydroxy-2-methyl-1H-indol-3-yl]acetic acid;
[1-(4-bromobenzyl)-5-hydroxy-2-methyl-1H-indol-3-yl]acetic acid;
[1-(4-chlorobenzyl)-5-methoxy-2-methyl-1H-indol-3-yl]acetic acid;
[1-(4-fluorobenzyl)-5-methoxy-2-methyl-1H-indol-3-yl]acetic acid;
[5-hydroxy-2-methyl-1-(3-methylbenzoyl)-1H-indol-3-yl]acetic acid;
{1-[(4-chlorophenyl)sulfonyl]-5-methoxy-2-methyl-1H-indol-3-yl}acetic acid;
{1-[(4-chlorophenyl)sulfonyl]-5-hydroxy-2-methyl-1H-indol-3-yl}acetic acid;
[5-methoxy-2-methyl-1-(piperidin-1-ylcarbonyl)-1H-indol-3-yl]acetic acid;
[5-hydroxy-2-methyl-1-(3-phenylprop-2-ynoyl)-1H-indol-3-yl]acetic acid;
propyl(5-hydroxy-2-methyl-1H-indol-3-yl)acetate; and
ethyl[1-(4-chlorobenzoyl)-5-methoxy-2-methyl-1H-indol-3-yl]acetate.

The invention also features a compound selected from:
(1-benzyl-5-hydroxy-2-methyl-1H-indol-3-yl)acetic acid;
[1-(3,4-dichlorobenzoyl)-5-hydroxy-2-methyl-1H-indol-3-yl]acetic acid;
[1-(3-chlorobenzoyl)-5-methoxy-2-methyl-1H-indol-3-yl]acetic acid;
[1-(3-chlorobenzyl)-5-methoxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
[1-(4-chlorobenzyl)-5-hydroxy-2-methyl-1H-indol-3-yl]acetic acid;
[1-(3,4-dichlorobenzoyl)-5-hydroxy-2-methyl-1H-indol-3-yl]acetic acid;
{5-hydroxy-2-methyl-1-[4-(trifluoromethyl)benzoyl]-1H-indol-3-yl}acetic acid;
[1-(2,4-dichlorobenzoyl)-5-hydroxy-2-methyl-H-indol-3-yl]acetic acid;
[1-(2,3-dichlorobenzoyl)-5-methoxy-2-methyl-1H-indol-3-yl]acetic acid;
[1-(2,3-dichlorobenzoyl)-5-hydroxy-2-methyl-1H-indol-3-yl]acetic acid;
{1-[(6-chloropyridin-3-yl)carbonyl]-5-hydroxy-2-methyl-1H-indol-3-yl}acetic acid;
[1-(3-chlorobenzoyl)-5-methoxy-2-methyl-1H-indol-3-yl]acetic acid;
[1-(3-chlorobenzoyl)-5-hydroxy-2-methyl-1H-indol-3-yl]acetic acid;
[1-(3,4-difluorobenzoyl)-5-methoxy-2-methyl-1H-indol-3-yl]acetic acid;
[1-(3,4-difluorobenzoyl)-5-hydroxy-2-methyl-1H-indol-3-yl]acetic acid;
[1-(4-bromobenzyl)-5-hydroxy-2-methyl-1H-indol-3-yl]acetic acid;
[5-hydroxy-2-methyl-1-(3-methylbenzoyl)-1H-indol-3-yl]acetic acid;
{1-[(4-chlorophenyl)sulfonyl]-5-hydroxy-2-methyl-1H-indol-3-yl}acetic acid;
[5-methoxy-2-methyl-1-(piperidin-1-ylcarbonyl)-1H-indol-3-yl]acetic acid;
[5-hydroxy-2-methyl-1-(3-phenylprop-2-ynoyl)-1H-indol-3-yl]acetic acid;
propyl(5-hydroxy-2-methyl-1H-indol-3-yl)acetate; and
ethyl[1-(4-chlorobenzoyl)-5-methoxy-2-methyl-1H-indol-3-yl]acetate.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, iodine and chlorine include ³H, ¹⁴C, ³⁵S, ³²P, ¹⁸F, ¹²⁵I and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of this invention and prodrugs thereof can generally be prepared by methods well known to those skilled in the art. Conveniently, such radiolabeled compounds can be prepared by carrying out the procedures disclosed in the examples of the instant specification by substituting a readily available radiolabeled reagent for a non-radiolabeled reagent.

In various embodiments: the cognitive function is short term memory, the cognitive function is long term memory, the patient is suffering from Alzheimer's Disease, the patient is suffering from mild cognitive impairment, and the patient is suffering from or at risk of developing impairment of cognitive function associated with treatment with a therapeutic agent. In other embodiments, the patient is suffering from a disorder selected from the group consisting of: vascular dementia, Huntington's Disease, hydrocephalus, depression, amnesia, AID-related dementia, Pick's Disease, Creutzfeldt-Jakob Syndrome, electroconvulsive therapy, Huntington's disease, amyotropic lateral sclerosis, dementia, stroke, mental retardation, Down syndrome, and Parkinson's Disease.

The compounds can be used to treat benign forgetfulness, a mild tendency to be unable to retrieve or recall information that was once registered, learned, and stored in memory. Benign forgetfulness typically affects individuals over 40 and can be recognized by standard assessment instruments such as the Wechsler Memory Scale (Russell, 1975, J. Consult Clin. Psychol. 43:800-809).

As note above, the compounds can be used for treating AD. Methods for diagnosing AD are known in the art. For example, the National Institute of Neurological and Communicative Disorders and Stroke-Alzheimer's Disease-and the Alzheimer's Disease and Related Disorders Association (NINCDS-ADRDA) criteria can be used to diagnose AD (McKhann et al. 1984 Neurology 34:939-944). The patient's cognitive function can be assessed by the Alzheimer's Disease Assessment Scale-cognitive subscale (ADAS-cog; Rosen et al., 1984, Am. J. Psychiatry 141:1356-1364).

The compounds can be used to treat neuropsychiatric disorders such as schizophrenia, autism, attention deficit disorder (ADD), and attention deficit-hyperactivity disorder (ADHD). They may be useful for treating mood disorders; anxiety related disorders; eating disorders; substance-abuse related disorders; personality disorders; and other mental disorders.

The compounds can be used to treat cognitive and memory impairment associated with head injury or trauma, sometimes referred to as amnesic disorder due to a general medical condition.

The invention includes a method for slowing or reducing cognitive impairment or memory loss or increasing cognitive function or memory by administering a composition comprising a compound having formula II.

In certain embodiments of the invention, the compound having formula II inhibits COX-2 activity, but does not substantially inhibit COX-1 activity. In other embodiments of the invention the compound having formula II inhibits COX-1 activity, but does not substantially inhibit COX-2 activity. In still other embodiments, the compound having formula II inhibits neither COX-1 activity nor COX-2 activity. For example, at a therapeutically relevant dosage the compound does not significantly inhibit either COX-1 or COX-2. Certain useful compound have an IC₅₀ for both COX-1 and COX-2 that is that is at least 3-, 5-, 10-, 20-, 100- or 500-fold greater than the IC₅₀ of indomethacin for both COX-1 and COX-2 in the same assay.

Certain compounds have an IC₅₀ for DAO that is at least 0.5, 2, 4, 10, 50, 100, 2000, 500, 1000-fold lower than indomethacin in the same assay.

In various methods of the invention, a patient is administered a composition comprising a compound having formula II and is not administered indomethacin.

The methods of the invention include treating a patient so as to achieve a serum level of a compound having formula II that is at least 10 nM.

In certain embodiments the composition comprising a compound having formula II does not include an inhibitor of COX-1, the composition does not include an inhibitor of COX-2, and the composition does not include an inhibitor of either COX-1 or COX-2.

The invention also features a pharmaceutical composition comprising a compound having formula II and a pharmaceutically acceptable carrier. The invention also features pharmaceutical composition comprising a compound having formula II, an agent for the treatment of memory loss (e.g., tacrine (Cognex®), donepezil hydrochloride (Aricept®D), galantamine (Reminyl®), rivastigmine (Exelon®), a cholinesterase inhibitor, an NMDA receptor antagonist (e.g., memantine), a M1 muscarinic receptor antagonist, vitamin E/tocopherol, a statin (e.g., lovastatin), CX516 (Ampalexg; Cortex Pharmaceuticals, Irvine, Calif.), aripipazole (Bristol-Meyers Squibb, Lawrenceville, N.J.), CPI-1189 (Centaur Pharmaceuticals, Sunnyvale, Calif.), leteprinim potassium (Neotrofin®; NeoTherapeutics, Inrine, Calif.), phenserine tartrate (Axonyx, New York, N.Y.), pravastatin (Pravachol®, Bristol-Meyers Squibb, Lawrenceville, N.J.), conjugated estrogen (Premain®; Wyeth, Philadelphia, Pa.), risperidone (Risperdal®, Johnson & Johnson Pharmaceutcals Research and Development, Raritan, N.J.), SB271046 (GlaxoSmithKline, Philadelphia, Pa.), SB737552 (GlaxoSmithKline, Philadelphia, Pa.), SR 57667 (Sanofi-Synthelabo, New York, N.Y.), and SR 57746 (Sanofi-Synthelabo, New York, N.Y.)) and a pharmaceutically acceptable carrier.

In certain embodiments the compounds are administered in combination with a second compound useful for slowing or reducing cognitive impairment or memory loss or increasing cognitive function or memory.

The compounds of the invention can be administered with D-serine or an analog thereof (e.g., a salt of D-serine, an ester of D-serine, alkylated D-serine, or a precursor of D-serine). They can administered with an anti-psychotic, an anti-depressant or a psychostimulant.

Treatments for depression can be used in combination with the compounds of the invention. Suitable anti-depressants include: tricyclic antidepressants (TCAs); monoamine oxidase inhibitors (MAOIs); serotonin selective reuptake inhibitors (SSRIs); dual serotonin and norepinephrine reuptake inhibitors; serotonin-2 antagonism/reuptake inhibitors; alpha₂/serotonin-2/seratonin-3 antagonists; and selective norepinephrine and dopamine reuptake inhibitors.

Anti-psychotic drugs can be used in combination with the compounds of the invention. Such treatments include: neuroleptics (e.g., chlorpromazine (Thorazine®); atypical neuroleptics (clozapine (Clozaril®)); risperidone (Risperdal®); and olanzapine (Zyprexa®).

Also within the invention are compounds having formula II that inhibit the activity of D-aspartate oxidase (DDO), an enzyme that oxidizes D-Asp, D-Glu, D-Asn, D-Gln, D-Asp-dimethyl-ester and N-methyl-D-Asp.

The compound of the invention can be administered in combination with a DAO or DDO inhibitor or antagonists such as those described in U.S. Application 20030166554, hereby incorporated by reference. Suitable DDO inhibitors can include: aminoethylcysteine-ketimine (AECK, thialysine ketimine, 2H-1,4-thiazine-5,6-dihydro-3-carboxylic acid, S-aminoethyl-L-cysteine ketimine, 2H-1,4-Thiazine-3-carboxylic acid, 5,6-dihydro-); aminoethylcysteine (thialysine); cysteamine; pantetheine; cystathionine; and S-adenosylmethionine.

The subject can be a mammal, preferably a human. Identifying a subject in need of such treatment can be in the judgment of a subject or a health care professional and can be subjective (e.g., opinion) or objective (e.g., measurable by a test or diagnostic method).

The term “treating” or “treated” refers to administering a compound described herein to a subject with the purpose to cure, heal, alleviate, relieve, alter, remedy, ameliorate, reverse, improve, or affect a disease, the symptoms of the disease, the recurrence of the disease, or the predisposition toward the disease.

“An effective amount” refers to an amount of a compound that confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, alternatively from about 1 to about 50 mg/Kg. Effective doses will also vary depending on route of administration, as well as the possibility of co-usage with other agents.

The term “mammal” includes, for example, mice, rats, cows, sheep, pigs, goats, and horses, monkeys, dogs, cats, rabbits, guinea pigs, Microcebus murinus, and primates, including humans.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Examples of “arylalkyl” or “aralkyl” include benzyl and 9-fluorenyl groups.

The terms “alkylamino” and “dialkylamino” refer to —NH(alkyl) and —N(alkyl)₂ radicals respectively. The term “aralkylamino” refers to a —NH(aralkyl) radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclopentyl, norbornyl, and adamantyl.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, acyloxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

Among the useful compounds useful in the methods of the invention are the compounds in FIG. 1 and: 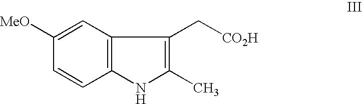
(5-methoxy-2-methyl-1H-indol-3-yl)acetic acid 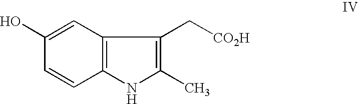
(5-hydroxy-2-methyl-1H-indol-3-yl)acetic acid 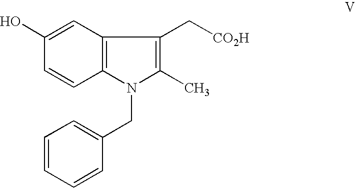
(1-benzyl-5-hydroxy-2-methyl-1H-indol-3-yl)acetic acid 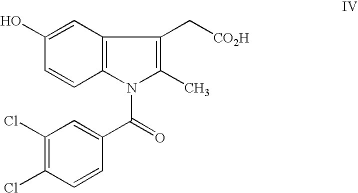
[1-(3,4-dichlorobenzoyl)-5-hydroxy-2-methyl-1H-indol-3-yl]acetic acid 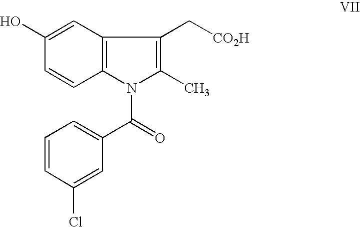
[1-(3-chlorobenzoyl)-5-methoxy-2-methyl-1H-indol-3-yl]acetic acid 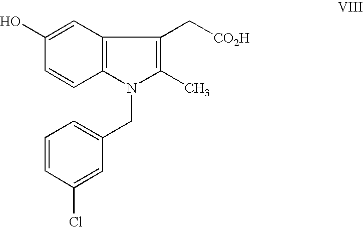
[1-(3-chlorobenzyl)-5-methoxy-2-methyl-1H-indol-3-yl]acetic acid 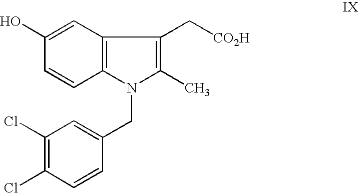
[1-(3,4-dichlorobenzoyl)-5-methoxy-2-methyl-H-indol-3-yl]acetic acid 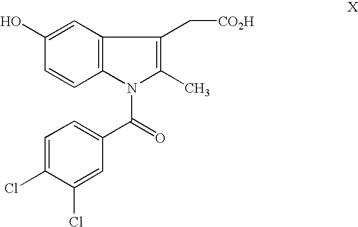
[1-(3,4-dichlorobenzoyl)-5-hydroxy-2-methyl-1H-indol-3-yl]acetic acid

Also useful are derivative Compounds III-X and derivatives of the compound in FIG. 1 in which the —COOH group has been converted to an ester, e.g., —COOR₁ wherein R₁ is alkyl or aryl or cycloaryl.

All diastereomeric forms possible (pure enantiomers, tautomers, racemic mixtures and unequal mixtures of two enantiomers) are within the scope of the invention. Such compounds can also occur as cis- or trans-, E- or Z-double bond isomer forms. All isomeric forms are contemplated.

The compounds described herein may be used as such or, where appropriate, as pharmacologically acceptable salts (acid or base addition salts) thereof.

The pharmacologically acceptable addition salts as mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

The details of one or more embodiments of the invention are set forth in the accompanying drawings and the description below. Other features, objects, and advantages of the invention will be apparent from the description and drawings, and from the claims.

### FIGURE

FIG. 1 depicts certain compounds along with their COX-1 IC₅₀ and their COX-2 IC₅₀.

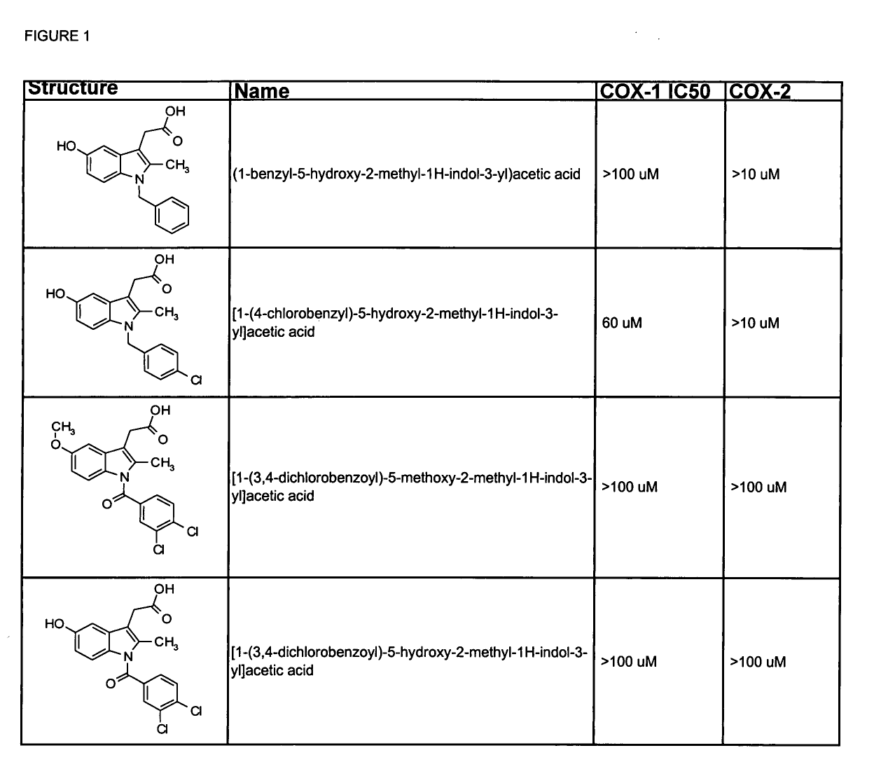

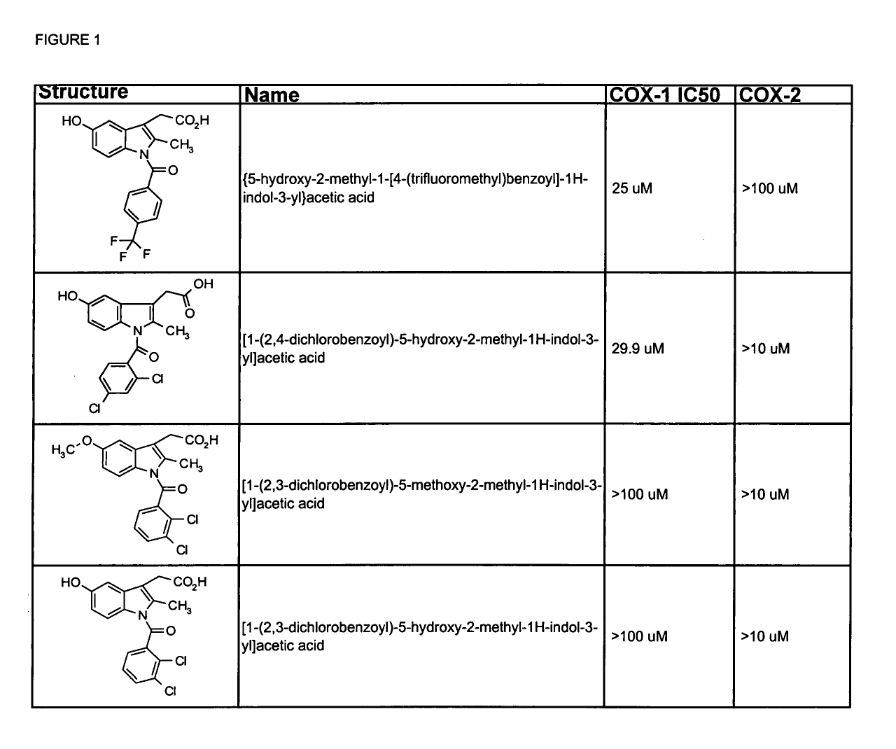

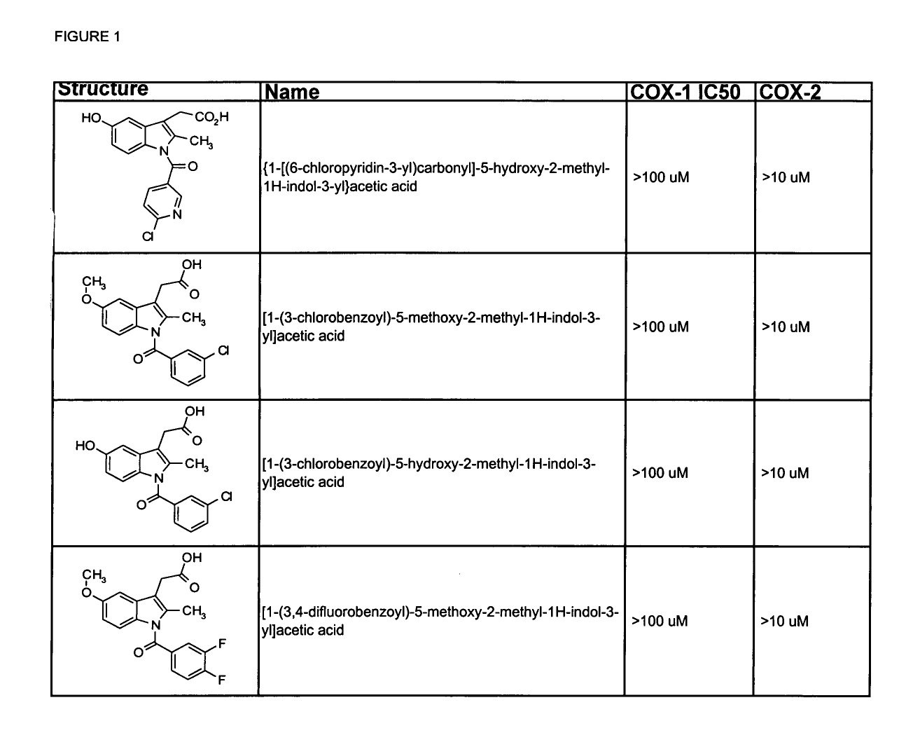

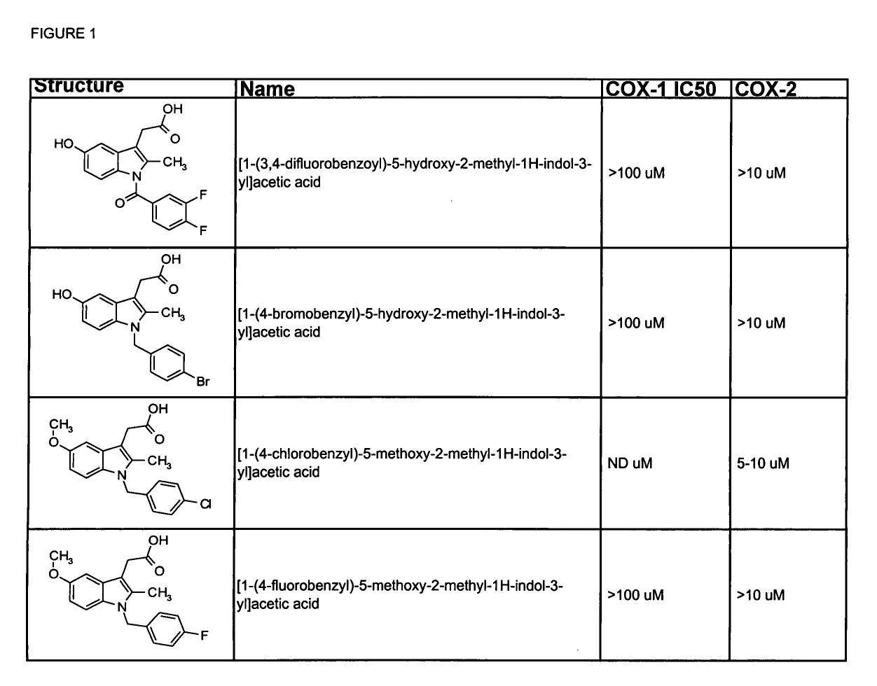

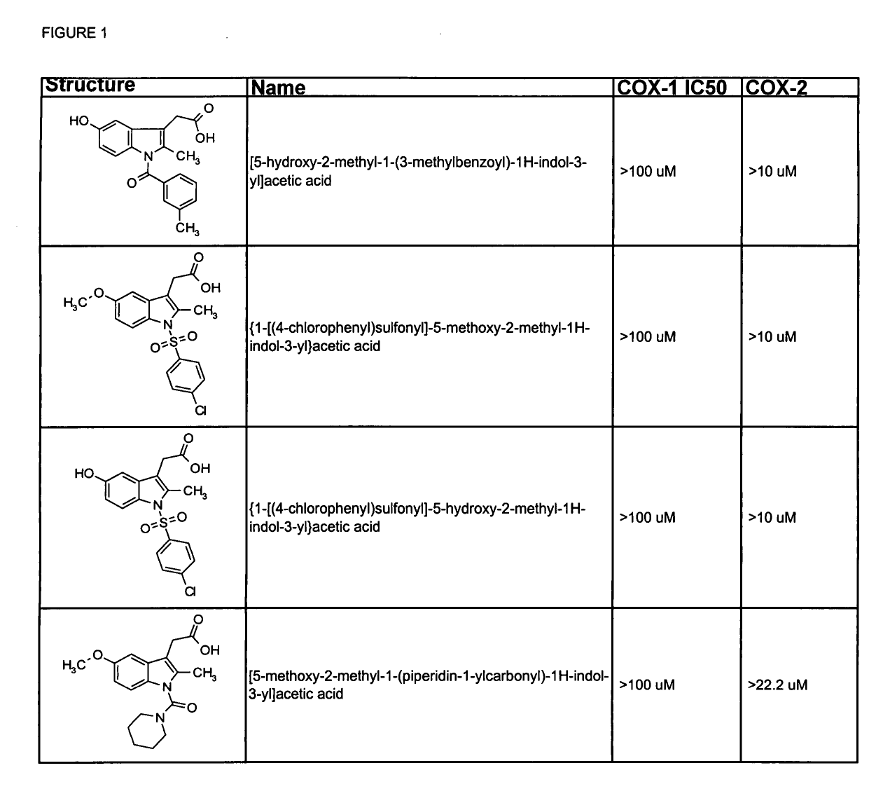

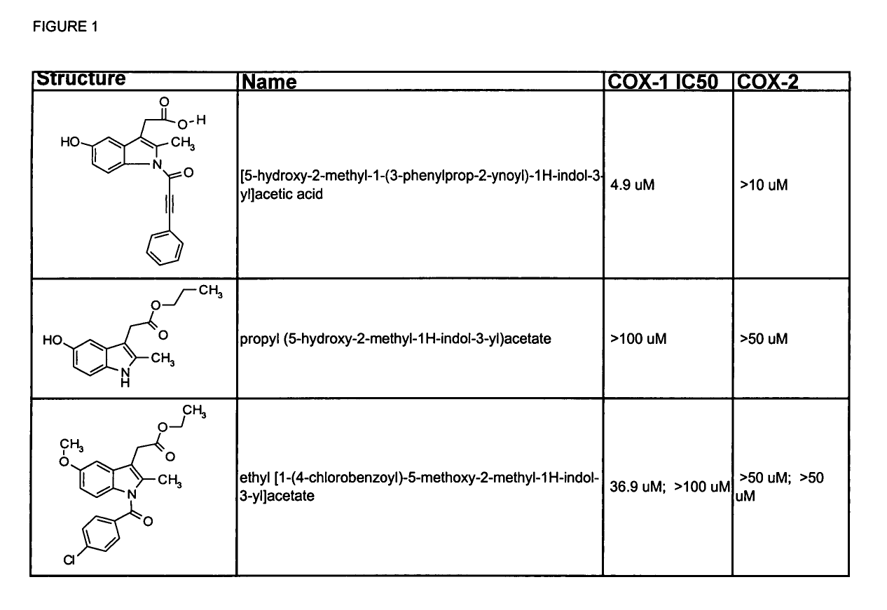

### DETAILED DESCRIPTION

Subsequent to indomethacin (I) (Shen et al. 1963 J. Am Chem. Soc. 85:488) administration, the unchanged parent compound, the desmethyl metabolite, the desbenzoyl metabolite and the desmethy-desbenzoyl metabolite can be found in plasma (Strachman et al. 1964 J. Am Chem. Soc. 8:799), all in their unconjugated forms (Harman et al. 1964 J. Pharmocol Exp Therap 143:215).

Useful metabolites and derivatives of indomethacin are those that inhibit one or more cyclooxygenases (e.g., COX-1 and COX-2) to a lesser extent than does indomethacin. Thus, the compounds have an IC₅₀ for COX-1 and/or COX-2 which is at least 2-, 5-, 10-, 15-, 20-, 100-, 500-, 1,000-fold greater than that of indomethacin. Particularly desirable are compounds that do not measurably inhibit COX-1 and/or COX-2.

The structure activity relationships of indomethacin derivatives have been established in the context of their ability to inhibit both COX-1 and COX-2 (Black et al. 1997 Advances in Experimental Medicine and Biology 407:73). In addition, excellent synthesis methodology has been demonstrated for the preparation of indomethacin analogues, some of which do not inhibit cyclooxygenases (Touhey et al. 2002 Eur J Cancer 38:1661). Thus, methods for the synthesis of the useful compounds are readily available.

Preparation of Compounds

In general, the compounds having formula II can be prepared according to the following scheme.

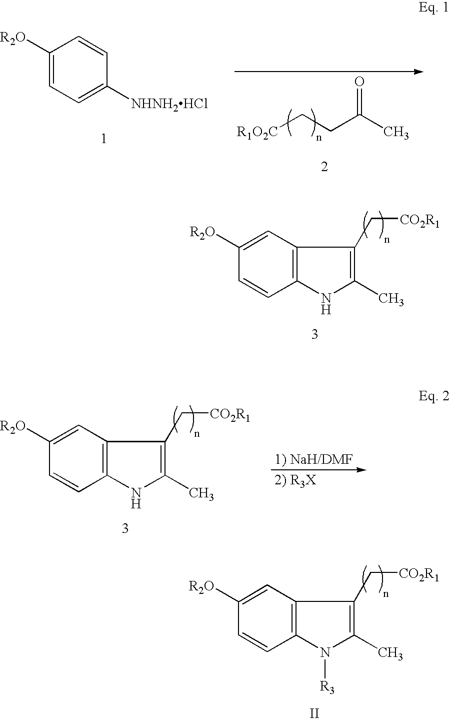

In this approach, a substituted phenyl hydrazine hydrochloride derivative (1) is condensed with an appropriately substituted ketoacid (2) in the presence of acid to provide the desired indole derivative (3) (see Eq. 1). In the second step the indole 3 is deprotonated with a strong base such as sodium hydride in a suitable solvent such as dimethylformamide (DMF) and then treated with an electrophile R₃X to provide the desired compounds (II) (see Eq. 2).

Administration of Therapeutic Compounds

The active compounds themselves can be administered to a patient or pro-drug form of the compound can be administered. The compounds can be administered before or after symptoms of cognitive deterioration or memory loss occur. They can be administered to healthy individuals to enhance cognitive function and/or to enhance memory.

The compounds of the invention can be used alone or in combination with other compounds used to treat, slow or prevent memory loss including known compounds for treatment of AD (e.g., memantine, galantamine, ravustigmine and donezepil). Combination therapies are useful in a variety of situations, including where an effective dose of one or more of the agents used in the combination therapy is associated with undesirable toxicity or side effects when not used in combination. This is because a combination therapy can be used to reduce the required dosage or duration of administration of the individual agents.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. The agents can be in the form of ammonium, calcium, magnesium, potassium, and sodium salts. Examples of salts derived from pharmaceutically acceptable organic non-toxic bases include salts of primary, secondary, and tertiary amines, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine. The agents can be in the form of tris salts.

The agents of the invention are can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents of the invention can also be administered by captisol delivery technology, rectal suppository or parenterally.

Compositions of the present invention may also optionally include other therapeutic ingredients, anti-caking agents, preservatives, sweetening agents, colorants, flavors, desiccants, plasticizers, dyes, and the like. Any such optional ingredient must be compatible with the compound of the invention to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents.

Binders include: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), and mixtures thereof.

Fillers include: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, and mixtures thereof.

Disintegrants which might be used include: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants which might be included in a pharaceutical formulation include: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W. R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), and mixtures thereof.

Anti-caking agents include: calcium silicate, magnesium silicate, silicon dioxide, colloidal silicon dioxide, talc, and mixtures thereof.

Antimicrobial agents include: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, and mixtures thereof.

Useful coating agents include: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, and mixtures thereof.

The agents either in their free form or as a salt can be combined with a polymer such as polylactic-glycolic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. Pat. No. 20030068384) to create a sustained release formulation. Such formulations can be used to implants that release a compound of the invention or another agent over a period of a few days, a few weeks or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. Pat. No. 2,003,0171296, U.S. Pat. No. 2,002,0176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and. US20020019446. In such sustained release formulations microparticles of compound are combined with microparticles of polymer. U.S. Pat. No. 6,011,011 and WO 94/06452 describe a sustained release formulation providing either polyethylene glycols (where PEG 300 and PEG 400 are most preferred) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. Pat. No. 20030064105, U.S. Pat. No. 2,003,0138488A1, U.S. Pat. No. 20030216307A1,U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299,WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered using high-velocity transdermal particle injection techniques using the hydrogel particle formulation described in U.S. Pat. No. 2,002,0061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranassaly using the formulation described in U.S. Pat. No. 5,179,079. Formulations suitable for parenteral injection are described in WO 00/62759. The agents can be administered using the casein formulation described in U.S. Pat. No. 20030206939 and WO 00/06108. The agents can be administered using the particulate formulations described in U.S. Pat. No. 20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccharide are advantageous in that some of them also reportedly enhance absorption of the compound in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. Pat. No. 2003019437, U.S. Pat. No. 20030165436, and WO 96/40089 (which includes vegetable oil). Sustained release formulations suitable for inhalation are described in U.S. Pat. No. 20010036481A1, 20030232019A1, and U.S. Pat. No. 20040018243A1 as well as in WO 01/13891, WO 02/067902, WO 03/072080, and WO 03/079885. Pulmonary formulations containing microparticles are described in WO 03/015750, U.S. Pat. No. 20030008013, and WO 00/00176. Pulmonary formulations containing stable glassy state powder are described in U.S. Pat. No. 20020141945 and U.S. Pat. No. 6,309,671. Other aerosol formulations are desribed in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. Pat. No. 20030053960 and WO 01/60341. The agents can be administered intranasally as described in U.S. Pat. No. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agent can be fused to immunoglobulins or albumin, or incorporated into a liposome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. Pat. No. 2,002,0055496, WO 00/47203, and U.S. Pat. No. 6,495,120. The agent can be delivered using nanoemulsion formulations described in WO 01/91728A2.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

Methods to increase chemical and/or physical stability of the agents the described herein are found in WO 00/04880, and WO 97/04796 and the references cited therein.

Methods to increase bioavailability of the agents described herein are found in U.S. Pat. No. 2,003,0198619, WO 01/49268, WO 00/32172, and WO 02/064166. Glycyrrhizinate can also be used as an absorption enhancer (see, e.g., EP397447). WO 03/004062 discusses Ulex europaeus I (UEAI) and UEAI mimetics which may be used to target the agents of the invention to the GI tract. The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

Identification of Compounds with Reduced COX-1 and/or COX-2 Inhibitory Activity

The invention features certain metabolites and derivatives of indomethacin having reduced COX-1 and/or COX-2 inhibitory activity. COX-1 and COX-2 convert arachidonic acid to PGH₂. By measuring the effect of a test compound PGH₂ production in the presence of COX-1 only, COX-2 only or both COX-1 and COX-2, one can assess the COX inhibitory activity of the test compound. The production of PGH₂ can be measured by reducing PGH₂ to PGF_{2α} with SnCl₂ and then detecting PGF_{2α} by EIA using a suitable antibody. Kits for the measurement of COX inhibitory activity are commercially available (Caymen Chemical; Ann Arbor, Mich.).

It can be useful to measure COX-1 and COX-2 activity in whole blood. To measure COX-1 activity in whole blood, 100 μl of whole blood from healthy human donors is combined with a 2 μl aliquot of test compound in vehicle or vehicle alone and incubated for 1 hr at 37° C. as described by Berg et al. (1999 Inflamm. Res. 48, 369-379). Serum is isolated from the sample by centrifugation at 12,000 g for 5 min at 4° C. and is assayed for thromboxane B2 (TXB2) levels using an ELISA assay (e.g., Cayman EIA Kit, Catalog Number 519031). To measure COX-2 activity in whole blood, 100 μl of heparinized whole blood from healthy human donors is combined with a 1 μl aliquot of 10 μg/ml LPS (lipopolysaccharide) and a 2 μl aliquot of test compound in vehicle or vehicle alone and incubated for 24 h at 37° C. as described by Berg et al. (supra). Serum is isolated from the sample by centrifugation at 12,000 g for 5 min at 4° C. and is assayed for PGE₂ using an ELISA assay (e.g., Cayman EIA Kit, Catalog Number 514010).

Identification of Compounds with DAO Inhibitory Activity

Porcine kidney D-amino acid oxidase and D-serine can be used to test the DAO inhibitory activity of compounds of the invention. The breakdown of D-serine by DAO produces hydrogen peroxidase, which can be measured using, for example, the Amplex®t Red Hydrogen Peroxide Assay Kit (Molecular Probes, Inc.; Eugene, Oreg.). Briefly, a working solution is prepared by mixing: sodium phosphate buffer (8.7 ml, 0.025M, pH 7.4), D-serine solution (1.0 ml, 100 mM in water), horseradish peroxidase (0.2 ml, 200 U/ml in buffer), and Amplex® Red solution (0.1 ml, 1 mg dye in 200 μl in DMSO (50 μM in DMSO)). A working enzyme solution is prepared by diluting a D-amino oxidase stock solution (65 U/ml) one hundred fold. The working solution (100 μl) is transferred to wells of microtiter plate and a solution of test compound is added. Next, 5 μl of the enzyme solution is added and the rate of hydrogen peroxide release is determined by measuring the oxidation of Amplex® Red by spectrophotometry (excitation wavelength 544 nm, emission wavelength, 590 nm) after a reaction time of five minutes.

Tests of Cognitive Ability

In human patients there are a number of tests that can be used to measure cognitive ability. Useful test include Mini-Mental State Examination (MMSE), Alzheimer's Disease Assessment Scale (ADAS), Boston Naming Test (BNT), and Token Test (TK). The test scores are generally analyzed by determining the percent increase or decrease over the test period compared to the baseline score at the beginning of the test period. These tests and others can be used to assess the effectiveness of the agents used for the treatment or prevention of cognitive impairment.

In analyzing candidate memory protective agents it can be useful to measure the effect of a test compound on the cognitive ability in an animal model. There are a wide range of such tests that can be used to assess candidate compounds.

One useful test involves the assessment of working memory/attention in mice. Briefly, the effect of a compound on spatial working memory can be characterized in aged mice (i.e. about 25 months old) and in young mice (i.e. about 3 months old). The working memory of the mice can first be compromised by pharmacological means (i.e. scopolamine-induced impairment). Working memory is the temporary storage of information (Bontempi et al. 2001 J Pharm and Exp Therap 299:297), and has been shown to be the primary type of memory disrupted in Alzheimer's disease, stroke and aging (Glasky et al. 1994 Pharm, Biochem and Behavior 47:325). Another useful test for assessing working memory measures Spontaneous Alternation behavior in mice. Spontaneous alternation is defined as the innate tendency of rodents to alternate free choices in a T-maze over a series of successive runs (Dember and Fowler 1958 Psychological Bulletin 55:412). This is a sequential procedure that relies on working memory because the ability to alternate requires that the animal retain specific information, which varies from trial to trial (Bontempi et al. 2003 Neuropsychopharmacology Apr. 2, 2003, 1-12). This test is also sensitive to varying parameters, such as delay intervals and increased number of trials, as well as pharmacological treatments affecting memory processes (Stefani and Gold, 2001 Journal of Neuroscience 21:609). In conducting this test, mice are first allowed to briefly explore a T-maze to become familiar with the apparatus. On the following day, a mouse is placed in a start box that is connected to the main stem of the T-maze. The elapsed time between the opening of the start box and the choice of an arm is measured (choice latency). The mouse is confined in the chosen arm for a set amount of time (e.g., 30 seconds) and then returned to the start box for the remaining consecutive trials in a testing session (Bontempi et al, 2003). Working memory performance for each mouse is assessed by the percentage of alternation over the trials in the testing session. Percentage is defined as entry in a different arm of the T-maze over successive trials.

The Delayed Non-Matching to Place (DNMTP) test is another useful animal model for testing the effect of a compound on cognitive ability. In this test, mice are trained and tested in an elevated eight-arm radial maze with a central start box placed in the center of a room with various pictures/objects placed around the room to serve as spatial cues. Each arm has a food pellet cup located at it far end. Food deprived animals are habituated to the apparatus with all arms open and baited over a couple of successive daily free exploration periods prior to the test day. The exploration period ceases when all arms are visited and all food pellets are consumed (Bontempi et al 2001, 2003). Animals are then trained to the DNMTP rule. A session consists of multiple trials that are separated by a defined interval. A trial consists of a study phase (two forced runs) and a test phase (two choice runs). In the study phase, the animal is given two consecutive forced runs in two different open arms. A forced run is when one arm of the maze opens allowing the animal to travel down to collect the food pellet and return to the central start box. After the second forced run, the test phase ensues. Two doors open simultaneously to begin the first choice run. One door reveals the first arm visited during the study phase and the other is an adjacent unvisited arm. Once the animal makes a choice and then returns to the start box, the next pair of doors open (second choice run). The second choice run consists of the second arm visited in the study phase and an adjacent novel arm. During the choice runs, the animal is reinforced only when it enters the arm that had not been previously visited during the study phase. This is the non-matching to place rule; the rule being not to return to a previously visited arm. Once a mouse is trained to the DNMTP rule, variable delay periods between the study and test phases can be introduced. Mice are allowed to adapt to the delay paradigm over a few consecutive days prior to compound testing. Compound testing is conducted over a several consecutive days followed by a washout period with no paradigm training, followed by a vehicle injection for measurement of baseline performance. Test compound or vehicle injections are acutely administered prior to the start of each testing session. Working memory is evaluated by the comparison of performance on drug days versus baseline days. The effects of putative cognitive enhancing drugs are commonly evaluated in the delayed non-matching to position task (Crawley, What's Wrong With My Mouse? Behavioral Phenotyping of Transgenic and Knockout Mice, Wiley-Liss, New York, 2000). The DNMTP task is similar to schedule-induced operant tasks which include delayed matching and delayed non-matching to position tests in automated chambers, generally used in rats (Bontempi et al., 2001; Crawley, 2000).

Working memory tests such as those described above are thought to require identification and use of novel information on each trial (predominately affecting attentional processes) whereas spatial reference memory tasks require the same information to be used across trials.

The Morris Water Maze Task is a spatial navigation task in which an animal uses visual clues to swim to a hidden platform. Animals to are motivated to find the fastest, most direct route to the platform in order to escape the water. The test typically consists of pre-training to a visible platform to test the animal's ability to conduct the procedural component of the task. Training for location of a hidden platform follows visible platform acquisition. Finally, a probe trial tests the animal's ability to find the spatial location that previously contained the hidden platform. Successful performance on the probe trial means that the animal spends significantly greater time in the trained quadrant versus non-trained quadrants. A deficit in learning and memory is defined as normal performance in the visible platform task but impaired performance on the hidden platform task.

Other tests, such as avoidance tasks, have been extensively used in the screening of compounds for cognitive enhancement (Crawley, 2000; Sarter et al. 1992 Psychopharmacology 107:461). For example, in the passive avoidance task, an animal is placed in a shuttle box containing a light and dark chamber (the dark is the natural preference of the rodent). The animal is trained to associate footshock with the properties of the natural preferred dark chamber. The next day, the animal is placed in the light chamber and latency to enter the dark chamber assesses the memory for the aversive association (Crawley, 2000). Potential drawbacks from these tests are that procedural components (the ability to acquire, store or retrieve memories) cannot be differentiated form declarative memory (remembering a specific item of information) as opposed to the Morris Water Maze task. Latency to enter the dark chamber on the first day is the only inherent control parameter in the avoidance task. It is known the passive avoidance task can be affected by fear because an animal is negatively affected by the footshock so the test is often used to complement other learning and memory assays (Yamaguchi et al. 2001 Jpn Journal of Pharmacology 87:240).

Tests of cognitive ability are generally used in conjunction with tests designed to rule out artifacts that would impair the animal from performing complex tasks. For example, general effects on motor function (hyperactivity or sedation) can be measured by testing locomotor activity, including stereotypy (Crawley, 2000). Motor coordination and balance can be assessed by assays such as the rotarod test. This test requires a mouse to continuously walk forward on a rotating cylinder to keep from falling off (Crawley, 2000).

A number of embodiments of the invention have been described. Nevertheless, it will be understood that various modifications may be made without departing from the spirit and scope of the invention.

1. A method for treating a patient comprising administering a composition comprising:
(a) compound having the formula: 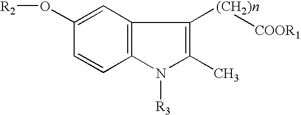
wherein:
R₁ is H or an independently substituted or unsubstituted C1-C10 alkyl, C3-C8 cycloalkyl, arylalkyl or heteroarylalkyl wherein the substituents are selected from the group consisting of amino, halogen and hydroxy;
n=1, 2, or 3; wherein each CH₂ within (CH₂)ₙ can be optionally independently substituted with one or more substituents selected from methyl, halogen and hydroxy;
R₂ is H or an independently substituted or unsubstituted C1-C4 alkyl or C3-C5 cycloalkyl wherein the substituents are selected from the group consisting of: methyl, halogen and hydroxy;
R₃ is H, —CH₂R₄, —C(O)R₄, —SO₂R₄, —CONR₅R₆ wherein:
R₄ is an independently substituted unsubstituted aryl or heteroaryl wherein the substituents are selected from halogen, OCH₃, OCF₂H, OCF₃, CH₃, CN, CF₂H, CF₃, SCH₃, SCF₃,
R₅ and R₆ are independently H, C1-C6 independently substituted or unsubstituted alkyl or R₅ together with R₆ form a 3 to 7-membered carbocyclic or heterocyclic ring wherein the heteroatoms are selected from O, S, SO, SO₂ and NR₇ wherein R₇ is H or an independently substituted or unsubstituted C1-C3 alkyl, wherein the substituents are selected from methyl, amino, halogen and hydroxy; and
(b) a pharmaceutically acceptable carrier.

2. The method of claim 1 wherein n is 1.

3. The method of claim 1 wherein n is 2.

4. The method of claim 1 wherein R₁ is H.

5. The method of claim 1 wherein R₁ is C1-C3 alkyl.

6. The method of claim 1 wherein R is methyl.

7. The method of claim 1 wherein R₃ is H.

8. The method of claim 1 wherein R₄ is a phenyl group.

9. The method of claim 8 wherein R₄ is unsubstituted.

10. The method of claim 8 wherein R₄ is substituted.

11. The method of claim 1 wherein R₃ is —CH₂R₄, or —C(O)R₄.

12. The method of claim 11 wherein R₄ is a phenyl group.

13. The method of claim 12 wherein the phenyl group is a substituted phenyl group.

14. The method of claim 13 wherein the phenyl group is independently substituted at the 3 and 4 positions.

15. The method of claim 13 wherein the phenyl group is independently substituted at the 2 and 4 positions.

16. The method of claim 13 wherein the phenyl group is independently substituted at the 2 and 3 positions.

17. The method of claim 13 wherein the phenyl group is substituted at the 4 position.

18. The method of claim 13 wherein the phenyl group is substituted at the 3 position.

20. The method of claim 13 wherein the phenyl group is independently substituted with one or more halogens.

21. The method of claim 13 wherein the phenyl group is substituted at the 4 position with CF₃.

22. The method of claim 1 wherein the patient is suffering from one or more disorders chosen from short term memory, loss of long term memory, Alzheimer's Disease, and mild cognitive impairment.

23. The method of claim 1 wherein the patient is suffering from or at risk of developing impairment of cognitive function associated with treatment with a therapeutic agent.

24. The method of claim 1 wherein the patient is suffering from one or more disorders chosen from: vascular dementia, Huntington's Disease, hydrocephalus, depression, amnesia, AIDS-related dementia, Pick's Disease, Creutzfeldt-Jakob Syndrome, and Parkinson's Disease.

25. The method of claim 1 wherein the patient has undergone electroconvulsive therapy.

26. The method of claim 1 wherein the compound is not:

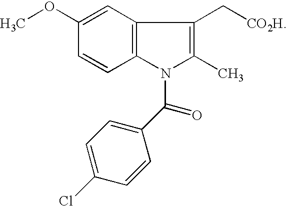

27. The method of claim 1 further comprising administering an agent chosen from: tacrine, donepezil hydrochloride, galantamine, rivastigmine, a cholinesterase inhibitor, an NMDA receptor antagonist, a M1 muscarinic receptor antagonist, vitamin E/tocopherol, a statin, CX516, aripipazole, CPI-1189, leteprinim potassium, phenserine tartrate, pravastatin, conjugated estrogen, risperidone, SB737552, SR 57667, and SR 57746.

28. The method of claim 1 wherein the compound does not substantially inhibit COX-1 activity.

29. The method of claim 1 wherein the compound does not substantially inhibit COX-2 activity.

30. The method of claim 1 wherein the compound does not substantially inhibit COX-1 activity or COX-2 activity.

31. The method of claim 1 wherein the compound is selected from:
(5-methoxy-2-methyl-1H-indol-3-yl)acetic acid;
(5-hydroxy-2-methyl-1H-indol-3-yl)acetic acid;
(1-benzyl-5-hydroxy-2-methyl-1H-indol-3-yl)acetic acid;
[1-(3,4-dichlorobenzoyl)-5-hydroxy-2-methyl-1H-indol-3-yl]acetic acid;
[1-(3-chlorobenzoyl)-5-methoxy-2-methyl-1H-indol-3-yl]acetic acid;
[1-(3-chlorobenzyl)-5-methoxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-H-indol-3-yl]acetic acid;
(1-benzyl-5-hydroxy-2-methyl-1H-indol-3-yl)acetic acid;
[1-(4-chlorobenzyl)-5-hydroxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
{5-hydroxy-2-methyl-1-[4-(trifluoromethyl)benzoyl]-1H-indol-3-yl}acetic acid;
[1-(2,4-dichlorobenzoyl)-5-hydroxy-2-methyl-1H-indol-3-yl]acetic acid;
[1-(2,3-dichlorobenzoyl)-5-methoxy-2-methyl-1H-indol-3-yl]acetic acid;
[1-(2,3-dichlorobenzoyl)-5-hydroxy-2-methyl-1H-indol-3-yl]acetic acid;
{1-[(6-chloropyridin-3-yl)carbonyl]-5-hydroxy-2-methyl-1H-indol-3-yl}acetic acid;
[1-(3-chlorobenzoyl)-5-methoxy-2-methyl-1H-indol-3-yl]acetic acid;
[1-(3-chlorobenzoyl)-5-hydroxy-2-methyl-1H-indol-3-yl]acetic acid;
[1-(3,4-difluorobenzoyl)-5-methoxy-2-methyl-1H-indol-3-yl]acetic acid;
[1-(3,4-difluorobenzoyl)-5-hydroxy-2-methyl-1H-indol-3-yl]acetic acid;
[1-(4-bromobenzyl)-5-hydroxy-2-methyl-1H-indol-3-yl]acetic acid;
[1-(4-chlorobenzyl)-5-methoxy-2-methyl-1H-indol-3-yl]acetic acid;
[1-(4-fluorobenzyl)-5-methoxy-2-methyl-1H-indol-3-yl]acetic acid;
[5-hydroxy-2-methyl-1-(3-methylbenzoyl)-1H-indol-3-yl]acetic acid;
{1-[(4-chlorophenyl)sulfonyl]-5-methoxy-2-methyl-1H-indol-3-yl}acetic acid;
{1-[(4-chlorophenyl)sulfonyl]-5-hydroxy-2-methyl-1H-indol-3-yl}acetic acid;
[5-methoxy-2-methyl-1-(piperidin-1-ylcarbonyl)-1H-indol-3-yl]acetic acid;
[5-hydroxy-2-methyl-1-(3-phenylprop-2-ynoyl)-1H-indol-3-yl]acetic acid;
propyl (5-hydroxy-2-methyl-1H-indol-3-yl)acetate; and
ethyl[1-(4-chlorobenzoyl)-5-methoxy-2-methyl-1H-indol-3-yl] acetate.

32. A pharmaceutical composition comprising:
(a) a compound having the formula: 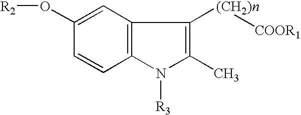
wherein:
R₁ is H or an independently substituted or unsubstituted C1-C10 alkyl, C3-C8 cycloalkyl, arylalkyl or heteroarylalkyl wherein the substituents are selected from the group consisting of amino, halogen and hydroxy;
n=1, 2, or 3; wherein each CH₂ within (CH₂)ₙ can be optionally independently substituted with one or more substituents selected from methyl, halogen and hydroxy;
R₂ is H or an independently substituted or unsubstituted C1-C4 alkyl or C3-C5 cycloalkyl wherein the substituents are selected from the group consisting of: methyl, halogen and hydroxy;
R₃ is H, —CH₂R₄, —C(O)R₄, —SO₂R₄, —CONR₅R₆ wherein:
R₄ is an independently substituted unsubstituted aryl or heteroaryl wherein the substituents are selected from halogen, OCH₃, OCF₂H, OCF₃, CH₃, CN, CF₂H, CF₃, SCH₃, SCF₃,
R₅ and R₆ are independently H, C1-C6 independently substituted or unsubstituted alkyl or R₅ together with R₆ form a 3 to 7-membered carbocyclic or heterocyclic ring wherein the heteroatoms are selected from O, S, SO, SO₂ and NR₇ wherein R₇ is H or an independently substituted or unsubstituted C1-C3 alkyl, wherein the substituents are selected from methyl, amino, halogen and hydroxy; and
(b) a pharmaceutically acceptable carrier.

33. The composition of claim 32 wherein n is 1.

34. The composition of claim 32 wherein n is 2.

35. The composition of claim 22 wherein R₁ is H.

36. The composition of claim 22 wherein R₁ is C1-C3 alkyl.

37. The composition of claim 22 wherein R₁ is methyl.

38. The composition of claim 22 wherein R₃ is H.

39. The composition of claim 22 wherein R₄ is a phenyl group.

40. The composition of claim 39 wherein R₄ is unsubstituted.

41. The composition of claim 39 wherein R₄ is substituted.

42. The composition of claim 32 wherein R₃ is —CH₂R₄, or —C(O)R₄.

43. The composition of claim 42 wherein R₄ is a phenyl group.

44. The composition of claim 43 wherein the phenyl group is a substituted phenyl group.

45. The composition of claim 44 wherein the phenyl group is independently substituted at the 3 and 4 positions.

46. The composition of claim 44 wherein the phenyl group is independently substituted at the 2 and 4 positions.

47. The composition of claim 44 wherein the phenyl group is independently substituted at the 2 and 3 positions.

48. The composition of claim 44 wherein the phenyl group is substituted at the 4 position.

49. The composition of claim 44 wherein the phenyl group is substituted at the 3 position.

50. The composition of claim 44 wherein the phenyl group is independently substituted with one or more halogens.

51. The composition of claim 44 wherein the phenyl group is substituted at the 4 position with CF₃.

52. The composition of claim 32 wherein the compound is not:

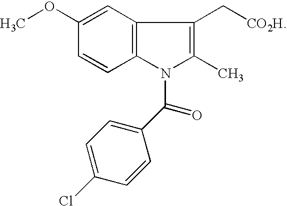

53. The composition of claim 32 further comprising an agent chosen from:
tacrine, donepezil hydrochloride, galantamine, rivastigmine, a cholinesterase inhibitor, an NMDA receptor antagonist, a M1 muscarinic receptor antagonist, vitamin E/tocopherol, a statin, CX516, aripipazole, CPI-1189, leteprinim potassium, phenserine tartrate, pravastatin, conjugated estrogen, risperidone, SB737552, SR 57667, and SR 57746.

54. The composition of claim 32 wherein the compound does not substantially inhibit COX-1 activity.

55. The composition of claim 32 wherein the compound does not substantially inhibit COX-2 activity.

56. The composition of claim 32 wherein the compound does not substantially inhibit COX-1 activity or COX-2 activity.

57. The method of claim 32 wherein the compound is selected from:
(5-methoxy-2-methyl-1H-indol-3-yl)acetic acid;
(5-hydroxy-2-methyl-1H-indol-3-yl)acetic acid;
(1-benzyl-5-hydroxy-2-methyl-1H-indol-3-yl)acetic acid;
[1-(3,4-dichlorobenzoyl)-5-hydroxy-2-methyl-1H-indol-3-yl]acetic acid;
[1-(3-chlorobenzoyl)-5-methoxy-2-methyl-1H-indol-3-yl]acetic acid;
[1-(3-chlorobenzyl)-5-methoxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
(1-benzyl-5-hydroxy-2-methyl-1H-indol-3-yl)acetic acid;
[1-(4-chlorobenzyl)-5-hydroxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
{5-hydroxy-2-methyl-1-[4-(trifluoromethyl)benzoyl]-1H-indol-3-yl}acetic acid;
[1-(2,4-dichlorobenzoyl)-5-hydroxy-2-methyl-1H-indol-3-yl]acetic acid;
[1-(2,3-dichlorobenzoyl)-5-methoxy-2-methyl-1H-indol-3-yl]acetic acid;
[1-(2,3-dichlorobenzoyl)-5-hydroxy-2-methyl-1H-indol-3-yl]acetic acid;
{1-[(6-chloropyridin-3-yl)carbonyl]-5-hydroxy-2-methyl-1H-indol-3-yl}acetic acid;
[1-(3-chlorobenzoyl)-5-methoxy-2-methyl-1H-indol-3-yl]acetic acid;
[1-(3-chlorobenzoyl)-5-hydroxy-2-methyl-1H-indol-3-yl]acetic acid;
[1-(3,4-difluorobenzoyl)-5-methoxy-2-methyl-1H-indol-3-yl]acetic acid;
[1-(3,4-difluorobenzoyl)-5-hydroxy-2-methyl-1H-indol-3-yl]acetic acid;
[1-(4-bromobenzyl)-5-hydroxy-2-methyl-1H-indol-3-yl]acetic acid;
[1-(4-chlorobenzyl)-5-methoxy-2-methyl-1H-indol-3-yl]acetic acid;
[1-(4-fluorobenzyl)-5-methoxy-2-methyl-1H-indol-3-yl]acetic acid;
[5-hydroxy-2-methyl-1-(3-methylbenzoyl)-1H-indol-3-yl]acetic acid;
{1-[(4-chlorophenyl)sulfonyl]-5-methoxy-2-methyl-1H-indol-3-yl}acetic acid;
{1-[(4-chlorophenyl)sulfonyl]-5-hydroxy-2-methyl-1H-indol-3-yl}acetic acid;
[5-methoxy-2-methyl-1-(piperidin-1-ylcarbonyl)-1H-indol-3-yl]acetic acid;
[5-hydroxy-2-methyl-1-(3-phenylprop-2-ynoyl)-1H-indol-3-yl]acetic acid;
propyl (5-hydroxy-2-methyl-1H-indol-3-yl)acetate; and
ethyl[1-(4-chlorobenzoyl)-5-methoxy-2-methyl-1H-indol-3-yl] acetate.

58. A method for enhancing cognitive function, comprising administering the pharmaceutical composition of claim 32.

59. The method of claim 58 wherein the compound does not substantially inhibit COX-1 activity.

60. The method of claim 58 wherein the compound does not substantially inhibit COX-2 activity.

61. The method of claim 58 wherein the compound does not substantially inhibit COX-1 activity or COX-2 activity.

62. The method of claim 1 or claim 58 wherein the compound is chosen from: (5-methoxy-2-methyl-1H-indol-3-yl) acetic acid; (5-hydroxy-2-methyl-1H-indol-3-yl)acetic acid; (5-hydroxy-2-methyl-1H-indol-3-yl)acetic acid; [1-(4-chlorobenzoyl)-5-methoxy-2-methyl-1H-indol-3-yl]acetic acid; (1-benzyl-5-hydroxy-2-methyl-1H-indol-3-yl)acetic acid; (1-benzoyl-5-methoxy-2-methyl-1H-indol-3-yl)acetic acid; [1-(3,4-dichlorobenzoyl)-5-hydroxy-2-methyl-1H-indol-3-yl]acetic acid; [1-(3-chlorobenzoyl)-5-methoxy-2-methyl-1H-indol-3-yl]acetic acid; [1-(3-chlorobenzyl)-5-methoxy-2-methyl-1H-indol-3-yl]acetic acid; (1-benzoyl-5-hydroxy-2-methyl-1H-indol-3-yl)acetic acid; [1-(3,4-dichlorobenzoyl)-5-methoxy-2-methyl-1H-indol-3-yl]acetic acid and salts thereof.

63. The pharmaceutical composition of claim 32 wherein the compound is chosen from: 5-methoxy-2-methyl-1H-indol-3-yl)acetic acid; (5-hydroxy-2-methyl-1H-indol-3-yl)acetic acid; (5-hydroxy-2-methyl-1H-indol-3-yl)acetic acid; [1-(4-chlorobenzoyl)-5-methoxy-2-methyl-1H-indol-3-yl]acetic acid; (1-benzyl-5-hydroxy-2-methyl-1H-indol-3-yl)acetic acid; (1-benzoyl-5-methoxy-2-methyl-1H-indol-3-yl)acetic acid; [1-(3,4-dichlorobenzoyl)-5-hydroxy-2-methyl-1H-indol-3-yl]acetic acid; [1-(3-chlorobenzoyl)-5-methoxy-2-methyl-1H-indol-3-yl]acetic acid; [1-(3-chlorobenzyl)-5-methoxy-2-methyl-1H-indol-3-yl]acetic acid; (1-benzoyl-5-hydroxy-2-methyl-1H-indol-3-yl)acetic acid; [1-(3,4-dichlorobenzoyl)-5-methoxy-2-methyl-1H-indol-3-yl]acetic acid and salts thereof.

64. The method of claim 58 wherein the compound is selected from:
(5-methoxy-2-methyl-1H-indol-3-yl)acetic acid;
(5-hydroxy-2-methyl-1H-indol-3-yl)acetic acid;
(1-benzyl-5-hydroxy-2-methyl-1H-indol-3-yl)acetic acid;
[1-(3,4-dichlorobenzoyl)-5-hydroxy-2-methyl-1H-indol-3-yl]acetic acid;
[1-(3-chlorobenzoyl)-5-methoxy-2-methyl-1H-indol-3-yl]acetic acid;
[1-(3-chlorobenzyl)-5-methoxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
(1-benzyl-5-hydroxy-2-methyl-1H-indol-3-yl)acetic acid;
[1-(4-chlorobenzyl)-5-hydroxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
{5-hydroxy-2-methyl-1-[4-(trifluoromethyl)benzoyl]-1H-indol-3-yl}acetic acid;
[1-(2,4-dichlorobenzoyl)-5-hydroxy-2-methyl-1H-indol-3-yl]acetic acid;
[1-(2,3-dichlorobenzoyl)-5-methoxy-2-methyl-1H-indol-3-yl]acetic acid;
[1-(2,3-dichlorobenzoyl)-5-hydroxy-2-methyl-1H-indol-3-yl]acetic acid;
{1-[(6-chloropyridin-3-yl)carbonyl]-5-hydroxy-2-methyl-1H-indol-3-yl}acetic acid;
[1-(3-chlorobenzoyl)-5-methoxy-2-methyl-1H-indol-3-yl]acetic acid;
[1-(3-chlorobenzoyl)-5-hydroxy-2-methyl-1H-indol-3-yl]acetic acid;
[1-(3,4-difluorobenzoyl)-5-methoxy-2-methyl-1H-indol-3-yl]acetic acid;
[1-(3,4-difluorobenzoyl)-5-hydroxy-2-methyl-1H-indol-3-yl]acetic acid;
[1-(4-bromobenzyl)-5-hydroxy-2-methyl-1H-indol-3-yl]acetic acid;
[1-(4-chlorobenzyl)-5-methoxy-2-methyl-1H-indol-3-yl]acetic acid;
[1-(4-fluorobenzyl)-5-methoxy-2-methyl-1H-indol-3-yl]acetic acid;
[5-hydroxy-2-methyl-1-(3-methylbenzoyl)-1H-indol-3-yl]acetic acid;
{1-[(4-chlorophenyl)sulfonyl]-5-methoxy-2-methyl-1H-indol-3-yl}acetic acid;
{1-[(4-chlorophenyl)sulfonyl]-5-hydroxy-2-methyl-1H-indol-3-yl}acetic acid;
[5-methoxy-2-methyl-1-(piperidin-1-ylcarbonyl)-1H-indol-3-yl]acetic acid;
[5-hydroxy-2-methyl-1-(3-phenylprop-2-ynoyl)-1H-indol-3-yl]acetic acid;
propyl (5-hydroxy-2-methyl-1H-indol-3-yl)acetate; and
ethyl[1-(4-chlorobenzoyl)-5-methoxy-2-methyl-1H-indol-3-yl] acetate.

65. A compound selected from:
(1-benzyl-5-hydroxy-2-methyl-1H-indol-3-yl)acetic acid;
[1-(3,4-dichlorobenzoyl)-5-hydroxy-2-methyl-1H-indol-3-yl]acetic acid;
[1-(3-chlorobenzoyl)-5-methoxy-2-methyl-1H-indol-3-yl]acetic acid;
[1-(3-chlorobenzyl)-5-methoxy-2-methyl-1H-indol-3-yl]acetic acid;
[1-(3,4-dichlorobenzoyl)-5-methoxy-2-methyl-1H-indol-3-yl]acetic acid;
[1-(3,4-dichlorobenzoyl)-5-hydroxy-2-methyl-1H-indol-3-yl]acetic acid;
[1-(4-chlorobenzyl)-5-hydroxy-2-methyl-1H-indol-3-yl]acetic acid;
[1-(3,4-dichlorobenzoyl)-5-hydroxy-2-methyl-1H-indol-3-yl]acetic acid;
{5-hydroxy-2-methyl-1-[4-(trifluoromethyl)benzoyl]-1H-indol-3-yl}acetic acid;
[1-(2,4-dichlorobenzoyl)-5-hydroxy-2-methyl-1H-indol-3-yl]acetic acid;
[1-(2,3-dichlorobenzoyl)-5-methoxy-2-methyl-1H-indol-3-yl]acetic acid;
[1-(2,3-dichlorobenzoyl)-5-hydroxy-2-methyl-1H-indol-3-yl]acetic acid;
{1-[(6-chloropyridin-3-yl)carbonyl]-5-hydroxy-2-methyl-1H-indol-3-yl}acetic acid;
[1-(3-chlorobenzoyl)-5-methoxy-2-methyl-1H-indol-3-yl]acetic acid;
[1-(3-chlorobenzoyl)-5-hydroxy-2-methyl-1H-indol-3-yl]acetic acid;
[1-(3,4-difluorobenzoyl)-5-methoxy-2-methyl-1H-indol-3-yl]acetic acid;
[1-(3,4-difluorobenzoyl)-5-hydroxy-2-methyl-1H-indol-3-yl]acetic acid;
[1-(4-bromobenzyl)-5-hydroxy-2-methyl-1H-indol-3-yl]acetic acid;
[5-hydroxy-2-methyl-1-(3-methylbenzoyl)-1H-indol-3-yl]acetic acid;
{1-[(4-chlorophenyl)sulfonyl]-5-hydroxy-2-methyl-1H-indol-3-yl}acetic acid;
[5-methoxy-2-methyl-1-(piperidin-1-ylcarbonyl)-1H-indol-3-yl]acetic acid;
[5-hydroxy-2-methyl-1-(3-phenylprop-2-ynoyl)-1H-indol-3-yl]acetic acid;
propyl (5-hydroxy-2-methyl-1H-indol-3-yl)acetate; and
ethyl[1-(4-chlorobenzoyl)-5-methoxy-2-methyl-1H-indol-3-yl] acetate.

66. A composition comprising a compound of claim 65 and a pharmaceutically acceptable carrier.
